(19) United States
(12) Patent Application Publication
(10) Pub. No.: US 2013/0034609 A1
(43) Pub. Date: Feb. 7, 2013

(54) SMART POLYMERS FUNCTIONALIZED HOLLOW SILICA VESICLES

(71) Applicants: Ye Liu, Singapore (SG); Chee Leng Lay, Singapore (SG)

(72) Inventors: Ye Liu, Singapore (SG); Chee Leng Lay, Singapore (SG)

(73) Assignee: Agency for Science, Technology and Research, Connexis (SG)

(21) Appl. No.: 13/577,860

(22) PCT Filed: Feb. 9, 2010
(86) PCT No.: PCT/SG2010/000043

§ 371 Date: Oct. 22, 2012

(51) Int. Cl. C08F 265/02; A61K 9/14; C08G 77/00; B32B 27/06; B82Y 5/00; B82Y 40/00; B82Y 30/00

(52) U.S. Cl. 424/489; 528/26; 525/102; 428/404; 977/897; 977/773; 977/906

(57) ABSTRACT

The present invention provides a porous hollow silica micro- or nanoparticle with a polymer grafted thereon, wherein the polymer is selected from poly(methacrylic acid) and copolymers thereof. The polymer may be covalently linked to the silica particle via a bridging group. Provided is also a method of covalently coupling a poly(methacrylic acid) to a silica surface of a hollow silica particle. The method comprises contacting a silica surface of a hollow silica particle that carries amino functional or halogen functional groups with a poly(methacrylic acid) or a copolymer or a respective monomer thereof. The method further comprises allowing the carboxyl group of the monomer or the poly(methacrylic acid) and an amino functional group or a halogen functional group on the silica surface to undergo a coupling reaction, thereby covalently coupling the polymer to the silica surface.

### FIELD OF THE INVENTION

The present invention relates to a porous hollow silica particle with a polymer grafted thereon. The invention further provides a method that is suitable for the formation of such a porous hollow silica particle.

### BACKGROUND OF THE INVENTION

Vesicles are formed by encapsulating a volume with a thin membrane which can be composed of lipids, polymers and hybrid materials. Vesicles are important for many applications such as for drug delivery with drugs being hold in the interiors or the membranes. Good vesicles should have 1) a good stability to get enough long shelf life for storage and good integrity for drug delivery before reaching target sites, 2) stealth layers to provide a good dispersity in aqueous solution and targeting capability, and 3) a suitable fluidity for release of species encapsulated at target sites.

However, the dilemma between stability and fluidity of vesicles always exists. Vesicles with good fluidity always have a poor stability. For example, liposomes obtained from self-assembly of amphiphilic lipids are dynamic and feasible for spices to move into and out, but stability of liposomes are poor due to the weak interaction among short hydrophobic lipid segments which is responsible for the integrity of liposomes. Cross-linking or formation of polymer based or silica cages can stabilize liposomes but also reduce the fluidity. In comparison, the structures and properties of polymer vesicles formed by self-assembly of amphiphilic copolymers can be adjusted in a wider range through tuning the chemistry, composition and molecular weight of copolymers. But stable polymer vesicles always have a low fluidity.

In order to obtain good vesicles, smart stable vesicles should be prepared. The smart stable vesicles have good integrity during storage and delivery process, and the release of the species encapsulated can be triggered by certain stimuli presented at the target sites. The stimuli can be temperature, pH, ionic strength, enzyme and light and so on.

Recently, nanosized hollow silica spheres were suggested as useful carriers of active species for encapsulation and controlled delivery because of their biocompatibility, large surface area and pore volume, adjustable pore diameter and modifiable surface properties. However hollow silica materials have fatal shortcomings. Easily aggregation of silica and poor dispersion in solvent hinder their applications. Poor processability of hollow silica results in heterogeneous products.

However, it is a formidable challenge to prepare smart stable vesicles. pH is one of the most important stimuli to trigger the release of encapsulated species from vesicles. Several types of pH responsive vesicles have been reported. pH responsive polymer vesicles can be obtained from specific polypeptides. Also pH responsive polymer vesicles can be obtained by integrating poly(acrylic acid) into the membranes of polymer vesicles. pH responsive liposomes are obtained from poly(acrylic acid) caged liposomes. However, these approaches need specific materials, and the stability of some vesicles still is problematic. Hence smart stable vesicles prepared by new approaches are desirable.

Suzuki et al. (Polym. Adv. Technol. (2000) 11, 92) reported that grafting poly(acrylic acid) is a further possibility to functionalize porous silica particles. Other reports have shown that mesoporous silica nanoparticles having poly(acrylic acid) grafted thereon have a pH-responsive shell for controlled uptake and release (Hong et al; J. Mater. Chem., (2009), 19, 5155). Also described are complex system using poly(methacrylic acid-co-vinyl triethoxylsilane) in mesoporous silica spheres to obtain a pH-responsive drug release (Gao et al., J. Phys. Chem. C 2009, 113, 12753-12758). However, these compounds are difficult to prepare or have difficult polymer chains as sensitive groups.

It is therefore an object of the present invention to provide a hollow particle with properties that overcome at least some of the above described disadvantages.

### SUMMARY OF THE INVENTION

In a first aspect the present invention provides a porous hollow silica particle with a polymer grafted thereon. The polymer, which is selected from poly(methacrylic acid) and copolymers thereof, is covalently linked, e.g. grafted, to the silica particle.

In a second aspect the invention provides a pharmaceutical composition. The pharmaceutical composition includes a plurality of hollow silica particles. Typically the hollow silica particles have an inner void that includes a pharmaceutically active compound.

In a third aspect the invention provides a method of covalently coupling a polymer to a silica surface. The method includes providing a silica surface. The silica surface carries amino functional groups or halogen functional groups. The method also includes providing a polymer. The polymer is selected from poly(methacrylic acid) and copolymers thereof. The method further includes contacting the polymer and the silica surface. The method also includes allowing the carboxy group of the polymer and an amino functional group or a halogen functional group on the silica surface to undergo a coupling reaction. By allowing this coupling reaction to occur, the polymer is covalently coupled to the silica surface.

The invention will be better understood with reference to the detailed description when considered in conjunction with the accompanying drawings.

### BRIEF DESCRIPTION OF THE DRAWINGS

FIG. 1 is a scheme of one exemplary preparation route of PMAA-graft-hollow silica vesicles according to the invention.

FIG. 2 depicts TGA curves of a) pristine hollow silica spheres; b) APS attached hollow silica spheres; c) bromide-functionalized hollow silica spheres; and d) PMAA-g-hollow silica vesicles.

FIG. 3 shows TEM images of a) pristine hollow silica spheres and b) PMAA-g-hollow silica spheres.

FIG. 4 depicts nitrogen adsorption-desorption isotherms for hollow silica spheres.

FIG. 5 illustrates ¹H NMR spectra of PMAA-g-hollow silica vesicles recorded in deuterium oxide at a pH of 7.4 and 3.4.

FIG. 6 depicts the pH sensitive mechanism of PMAA-graft-hollow silica spheres.

FIG. 7 shows exemplary the loading and release mechanism of the PMAA-g-hollow silica spheres of the invention.

FIG. 8 shows the release profile of Calcein blue from a) PMAA-g-hollow silica vesicles and b) free PMAA at pH 2.0 and 7.4, respectively.

FIG. 9 shows the release profile of FTIC-dextran (Mw: 10 K) from a) PMAA-g-hollow silica vesicles and b) free PMAA at pH 2.0 and 7.4, respectively.

### DETAILED DESCRIPTION OF THE INVENTION

The present invention is based on the finding that poly(methacrylic acid) or copolymers thereof grafted onto a silica surface may alter the properties of the overall system. In doing so an intelligent, pH-sensitive nanoshell onto the exterior surface of hollow silica particles can be obtained. When grafted on hollow silica vesicles, the porous silica shells provide the vesicles stability and feasibility to load and release of guest molecules while the pH-responsive polymer shell can be reversibly opened and closed triggered by pH, which can act as a valve to regulate the loading and release of guest molecules from the silica core. These and other advantages will become more readily apparent from the following explanations and the appended drawings.

The poly(methacrylic acid) is linked to the surface of the hollow silica particle. The poly(methacrylic acid) may be selected from compounds according to general formula (1)

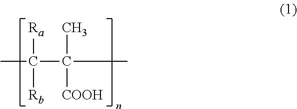

wherein Rₐ and R_{b} are independently an aliphatic, an alicyclic, an aromatic and an arylaliphatic group with a main chain of about 1 to about 30 carbon atoms and 0 to about 10 heteroatoms selected from the group consisting of N, O, S, Se and Si.; and n is an integer from 2 to 1000; wherein all groups may be optionally substituted

The aliphatic, alicyclic, aromatic or arylaliphatic group has a main chain of 1 to about 30 carbon atoms such as 2 to about 30 carbon atoms or 2 to about 25 carbon atoms, including about 1 to about 20 carbon atoms, about 2 to about 20 carbon atoms, about 3 to about 20 carbon atoms, about 1 to about 15 carbon atoms, about 2 to about 15 carbon atoms, about 1 to about 10 carbon atoms, about 2 to about 10 carbon atoms, about 1 to about 10 carbon atoms, or about 1 to about 6 carbon atoms, such as 1, 2, 3, 4, 5, 6, 7, 8, 9, 10, 11, 12, 13, 14, 15, 16, 17, 18, 19 or 20 carbon atoms. The main chain of the aliphatic, alicyclic, aromatic or arylaliphatic group may include 0 to about 10 heteroatoms, such as 0 to about 8 heteroatoms, 0 to about 7 or 0 to about 6, e.g. 0 to about 5 or 0 to about 4, including 0, 1, 2, 3, 4, 5 or 6 heteroatoms. A heteroatom is any other atom than carbon and hydrogen, such as N, O, S, Se and Si, but not limited to.

The term “aliphatic” means, unless otherwise stated, a straight or branched hydrocarbon chain, which may be saturated or mono- or poly-unsaturated and include heteroatoms. The term “heteroatom” as used herein means an atom of any element other than carbon or hydrogen. An unsaturated aliphatic group may contain one or more double and/or triple bonds (alkenyl or alkinyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1 to 5, to 10, to 15 or to 20 carbon atoms. Examples of alkenyl radicals are straight-chain or branched hydrocarbon radicals which contain one or more double bonds. Alkenyl radicals generally contain about two to about twenty carbon atoms and one or more, for instance two, double bonds, such as about two to about ten carbon atoms, and one double bond. Alkynyl radicals normally contain about two to about twenty carbon atoms and one or more, for example two, triple bonds, preferably such as two to ten carbon atoms, and one triple bond. Examples of alkynyl radicals are straight-chain or branched hydrocarbon radicals which contain one or more triple bonds. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, text-butyl, neopentyl, or 3,3-dimethylbutyl. Both the main chain as well as the branches may furthermore contain heteroatoms as for instance N, O, S, Se or Si or carbon atoms may be replaced by these heteroatoms.

The term “alkoxy”, alone or in combination, refers to an aliphatic hydrocarbon having an alkyl-O-moiety. In certain embodiments, alkoxy groups are optionally substituted. Typically, the hydrocarbon (main) chain includes 1 to 5, to 10, to 15 or to 20 carbon atoms such as 1, 2, 3, 4, or 5 carbon atoms. Examples of alkoxy groups include, but are not limited to, methoxy, ethoxy, propoxy, butoxy and the like.

The term “alicyclic” may also be referred to as “cycloaliphatic” and means, unless stated otherwise, a non-aromatic cyclic moiety (e.g. hydrocarbon moiety), which may be saturated or mono-or poly-unsaturated. The cyclic hydrocarbon moiety may also include fused cyclic ring systems such as decalin and may also be substituted with non-aromatic cyclic as well as chain elements. The main chain of the cyclic hydrocarbon moiety may, unless otherwise stated, be of any length and contain any number of non-aromatic cyclic and chain elements. Typically, the hydrocarbon (main) chain includes 3, 4, 5, 6, 7 or 8 main chain atoms in one cycle. Examples of such moieties include, but are not limited to, cyclopentyl, cyclohexyl, cycloheptyl, or cyclooctyl. Both the cyclic hydrocarbon moiety and, if present, any cyclic and chain substituents may furthermore contain heteroatoms, as for instance N, O, S, Se or Si, or a carbon atom may be replaced by these heteroatoms. The term “alicyclic” also includes cycloalkenyl moieties that are unsaturated cyclic hydrocarbons, which generally contain about three to about eight ring carbon atoms, for example five or six ring carbon atoms. Cycloalkenyl radicals typically have a double bond in the respective ring system. Cycloalkenyl radicals may in turn be substituted. Examples of such moieties include, but are not limited to, cyclohexenyl, cyclooctenyl or cyclodecenyl.

In contrast thereto, the term “aromatic” means an at least essentially planar cyclic hydrocarbon moiety of conjugated double bonds, which may be a single ring or include multiple condensed (fused) or covalently linked rings, for example, 2, 3 or 4 fused rings. The term aromatic also includes alkylaryl. Typically, the hydrocarbon (main) chain includes 5, 6, 7 or 8 main chain atoms in one cycle. Examples of such moieties include, but are not limited to, cyclopentadienyl, phenyl, napthalenyl-, [10]annulenyl-(1,3,5,7,9-cyclodecapentaenyl-), [12]annulenyl-, [8]annulenyl-, phenalene (perinaphthene), 1,9-dihydropyrene, chrysene (1,2-benzophenanthrene). An example of an alkylaryl moiety is benzyl. The main chain of the cyclic hydrocarbon moiety may, unless otherwise stated, be of any length and contain any number of heteroatoms, as for instance N, O and S. Such a heteroaromatic moiety may for example be a 5- to 7-membered unsaturated heterocycle which has one or more heteroatoms from the series O, N, S. Examples of such heteroaromatic moieties (which are known to the person skilled in the art) include, but are not limited to, furanyl-, thiophenyl-, naphtyl-, naphthofuranyl-, anthrathiophenyl-, pyridinyl-, pyrrolyl-, quinolinyl, naphthoquinolinyl-, quinoxalinyl-, indolyl-, benzindolyl-, imidazolyl-, oxazolyl-, oxoninyl-, oxepinyl-, benzoxepinyl-, azepinyl-, thiepinyl-, selenepinyl-, thioninyl-, azecinyl-, (azacyclodecapentaenyl-), diazecinyl-, azacyclododeca-1,3,5,7,9,11-hexaene-5,9-diyl-, azozinyl-, diazocinyl-benzazocinyl-, azecinyl-, azaundecinyl-, thia[11]annulenyl-, oxacyclotrideca-2,4,6,8,10,12-hexaenyl- or triaza-anthracenyl-moieties.

By the term “arylaliphatic” is meant a hydrocarbon moiety, in which one or more aromatic moieties are substituted with one or more aliphatic groups. Thus the term “arylaliphatic” also includes hydrocarbon moieties, in which two or more aryl groups are connected via one or more aliphatic chain or chains of any length, for instance a methylene group. Typically, the hydrocarbon (main) chain includes 5, 6, 7 or 8 main chain atoms in each ring of the aromatic moiety. Examples of arylaliphatic moieties such as alkylaryl moieties include, but are not limited, to 1-ethyl-naphthalene, 1,1′-methylenebis-benzene, 9-isopropylanthracene, 1,2,3-trimethyl-benzene, 4-phenyl-2-buten-1-ol, 7-chloro-3-(1-methylethyl)-quinoline, 3-heptyl-furan, 6-[2-(2,5-diethylphenyl)ethyl]-4-ethyl-quinazoline or, 7,8-dibutyl-5,6-diethyl-isoquinoline.

Each of the terms “aliphatic”, “alicyclic”, “alkoxy”, “aromatic” and “arylaliphatic” as used herein is meant to include both substituted and unsubstituted forms of the respective moiety. Substituents may be any functional group, as for example, but not limited to, amino, amido, azido, carbonyl, carboxyl, cyano, isocyano, dithiane, halogen, hydroxyl, nitro, organometal, organoboron, seleno, silyl, silano, sulfonyl, thio, thiocyano, trifluoromethyl sulfonyl, p-toluenesulfonyl, bromobenzenesulfonyl, nitrobenzenesulfonyl, and methanesulfonyl.

Referring to the above formula (1), in one embodiment Rₐ and R_{b} may be selected from the group consisting of hydrogen, methyl, ethyl and propyl, for example both Rₐ and R_{b} may be hydrogen.

n is an integer from 1 to about 10000, such as 1 to about 9000, 1 to about 8000, 1 to about 7000 1 to about 6000, 1 to about 5000, 1 to about 4000, 1 to about 3000 and 1 to about 2000. n may also be any integer not explicitly mentioned in the above listing.

The polymer may be attached to the silica surface as final polymer molecule. Alternatively, in another embodiment of the invention a monomer molecule may be attached to the silica surface. A polymerization reaction may subsequently be conducted in order to polymerize the monomer with further monomers to obtain the final polymer chain at the silica surface. Such polymerization reaction may be carried out under conditions suitable to prepare a poly(methacrylic acid) or a copolymer thereof. In this respect the term “copolymer” means that the final polymer chain (made before or after attachment to the silica surface) may be made from different monomers, i.e. monomers that have different substituents Rₐ and R_{b} in the above formula (1) and further monomers. By using copolymers a further modification of the polymer chain is possible. If desired, it is also possible in the present invention to combine polymerization of monomers and attaching a pre-formed polymer in order to graft the poly(methacrylic acid) polymer on the hollow silica particles.

Besides the monomers having different substituents Rₐ and R_{b}, in one embodiment of the present invention further monomers may be selected from vinyl monomers of the general formula (3) CH₂═CRₓR_{y}. In formula (3) Rₓ and R_{y} can each be independently selected from the group consisting of H, optionally substituted aliphatic, an alicyclic, an aromatic and an arylaliphatic group with a main chain of about 1 to about 30 carbon atoms and 0 to about 10 heteroatoms selected from the group consisting of N, O, S, Se and Si. Examples of such vinyl monomers include, but are not limited to, 1,3-butadiene, isoprene, styrene, [α]-methyl styrene, divinyl benzene, acrylonitrile, methacrylonitrile, vinyl halides such as vinyl chloride, vinyl esters such as vinyl acetate, vinyl propionate, vinyl laurate, and vinyl esters of versatic acids, heterocyclic vinyl compounds, alkyl esters of mono-olefinically unsaturated dicarboxylic acids (such as di-n-butyl maleate and di-n-butyl fumarate, fumaric acid, maleic acid, and itaconic acid, and optionally substituted alkyl esters of 1 to 20 carbon atoms thereof.

In one embodiment of the present invention, the vinyl monomer may include an acrylic monomer, such as, but not limited to, methyl(meth)acrylate, ethyl(meth)acrylate, n-butyl(meth)acrylate, 2-ethylhexyl(meth)acrylate, isopropyl(meth)acrylate, n-propyl(meth)acrylate, and hydroxyalkyl(meth)acrylates such as hydroxyethyl(meth)acrylate and 2-hydroxypropyl(meth)acrylate. In the following each vinyl monomer will be referred to as “vinyl”.

Representing the hollow silica particle by the symbol

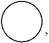

the entire particle with the polymer immobilized thereon can thus be depicted as (simplified illustration):

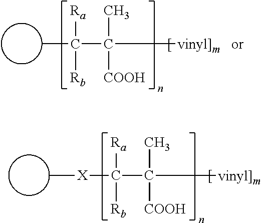

wherein X may be a bridging group linking the polymer to the silica surface. The index m may be an integer from 1 to about 10000, such as 1 to about 9000, 1 to about 8000, 1 to about 7000 1 to about 6000, 1 to about 5000, 1 to about 4000, 1 to about 3000 and 1 to about 2000. The index m may also be any integer not explicitly mentioned in the above listing. Typically the polymer defines a coating on the surface of the hollow silica particle. In one embodiment of the invention the polymer may be a linear, i.e. straight polymer. In some embodiments it may be a hyperbranched polymer. The polymer will usually have a molecular weight in the range from about 500-1.000.000, such as about 500-500.000, 500-200.000, 500-100.000, 500-50.000, 500-25.000, 500-10.000 or 500-5.000. It should be noted that in the above formulas the order of the monomers is only exemplary, i.e. the order may be changed. Further, it should also be noted that the monomers may be present in different combinations, i.e. as alternating copolymers, periodic copolymers, statistical copolymers and/or block copolymers, wherein alternating copolymers are copolymers with regular alternating A and B units; periodic copolymers have A and B units arranged in a repeating sequence (e.g. (A-B-A-B-B-A-A-A-A-B-B-B)ₙ), statistical copolymers are copolymers in which the sequence of monomer residues follows a statistical rule, and block copolymers comprise two or more homopolymer subunits linked by covalent bonds. The union of the homopolymer subunits may require an intermediate non-repeating subunit, known as a junction block. Block copolymers with two or three distinct blocks are called diblock copolymers and triblock copolymers, respectively. In one embodiment three or more different copolymers may be used, wherein one of the monomers has to be methacrylic acid.

As mentioned above, on the silica surface on which the polymer is immobilized, in the present case the surface of the hollow silica particle, the polymer has a particular high density. The surface of the silica particle may for example have a polymer content/polymer density in the range from about 10% to about 90% (w/w), such as about 10% to about 80% (w/w), about 15% to about 90% (w/w), about 15% to about 80% (w/w), about 20% to about 90% (w/w), about 20% to about 80% (w/w), about 20% to about 70% (w/w), about 15% to about 70% (w/w) or about 20% to about 60% (w/w) or about 30% to about 80% (w/w). Typically the surface has a polymer content/polymer density of at least about 20% (w/w), including at least about 25% (w/w), at least about 30% (w/w), at least about 35% (w/w), at least about 40% (w/w), or at least about 45% (w/w).

A particle according to the invention has typically a maximal width of about 1 nm to about 100 μm, such as about 1 nm to about 50 μm, about 1 nm to about 10 μm, about 2 nm to about 10 μm, about 2 nm to about 500 nm, about 2 nm to about 250 nm, about 10 nm to about 500 nm, about 10 nm to about 250 nm, about 25 nm to about 250 nm or about 50 nm to about 250 nm, such as about 250 nm, about 200 nm, about 190 nm or about 150 nm. The particle may be of any shape and geometry, including ball-shaped, i.e. spherical, rod shape, the shape of a disc or the shape of a rope.

The (in some embodiments water-soluble) microparticle or nanoparticle is typically a hollow micro- or nanosphere, i.e. it includes a void or cavity. In typical embodiments the microparticle or nanoparticle may optionally have a shell surrounding a void. The shell may be defined by a single wall with an internal and an external surface (i.e., balloon-like). The void or cavity may include the same fluid as the ambient fluid that surrounds the particle. The wall defining the shell may in some embodiments be considered to be an at least essentially closed and contiguous surface, in which some cracks and/or blowholes can occur. The wall defining the shell may in some embodiments have a thickness of about 0.1 nm to about 1000 nm, such as about 1 nm to about 500 nm, about 1 nm to about 250 nm, about 2 nm to about 50 nm, about 5 nm to about 50 nm, about 2 nm to about 25 nm or about 5 nm to about 20 nm, such as about 10, 12, 14, 15 or about 20 nm.

In some embodiments the particle, for example within a shell thereof, has one or more pores via which the void or cavity is in fluid contact with the ambience. Accordingly, the particle may be microporous or mesoporous. Microporous matter is in the art understood to have pores of a width of less than about 2 nm, whereas mesoporous matter is understood as having pores of about 2 nm to about 50 nm. The particle may for instance have a porous shell with a pore width of about 0.1 nm to about 500 nm, including a pore width of about 0.1 nm to about 8 nm, of about 0.5 nm to about 6 nm, of about 1 nm to about 6 nm, of about 1 nm to about 10 nm, about 1 nm to about 50 nm, about 1 nm to about 100 nm, about 1 nm to about 250 nm, about 1 nm to about 500 nm, such as a pore width of about 1 nm. The pore volume of the particle is in some embodiments selected in the range from about 0.01 cm³/g to about 2 cm³/g, such as from about 0.05 cm³/g to about 2 cm³/g, from about 0.05 cm³/g to about 1 cm³/g, from about 0.1 cm³/g to about 1 cm³/g or from about 0.1 cm³/g to about 0.5 cm³/g, e.g. about 0.25 cm³/g. A respective mesoporous or microporous particle may have any form and shape, including a sphere, a rod, a disc or a rope. The pores may be arranged in an ordered arrangement with symmetry such as hexagonal, cubic or lamellar.

The pores of the particle can be analysed by a variety of techniques. Examples include, but are not limited to, transmission electron microscopy, scanning electron microscopy, gas, e.g. nitrogen, adsorption, inverse platinum replica imaging, small-angle X-ray scattering, small-angle neutron scattering and positron annihilation lifetime spectroscopy. In some embodiments of transmission electron microscopy (TEM) a series of TEM images is taken from the same position at different tilt angles and 3D-information obtained in the so called tomography mode. In some embodiments of scanning electron microscopy (SEM) high resolution-SEM is used, working at very low currents and voltages. Structural information can furthermore be taken from NMR, Raman and FTIR spectroscopies, electrochemical methods, UV-Vis absorption and fluorescence spectroscopies, as well as single molecule spectroscopic methods. In single molecule spectroscopic methods the materials are typically investigated by doping them with very low, usually nanomolar concentrations of fluorescent dyes. Individual molecules and/or individual nanoscale environments can then be analysed.

The wall of the particle, if present, may be of small or even negligible thickness in comparison to the particle dimensions, such as less than about 10%, less than about 5%, less than about 2% or less than about 1% of the maximal particle width. The wall defining the shell may be microporous (e.g. sponge-like) in nature. Further matter may be included in the respective void or cavity, such as a fluid, including a liquid. A respective fluid included within the particle may in some embodiments include or be a pharmaceutically active compound and/or an excipient. The pharmaceutically active compound may be a low molecular weight organic compound. In some embodiments the pharmaceutically active compound is or includes a peptide, a protein, a lipid, a saccharide or a polysaccharide. The pharmaceutically active compound may be more or less homogenously distributed, e.g. dispersed, within the water-soluble microparticle or nanoparticle. In some embodiments the pharmaceutically active compound is located within a certain portion of the water-soluble microparticle or nanoparticle, such as a core or a shell.

The particle may have any surface area which is suitable for the present invention. For example, the particle may have a BET surface area in the range from about 10 m2/g to about 1000 m2/g, such as about 25 m2/g to about 500 m2/g, about 50 m2/g to about 250 m2/g, about 75 m2/g to about 200 m2/g or about 100 m2/g to about 200 m2/g, including a BET surface area of about 170 m2/g or about 160 m2/g. Other surface area are possible.

On the external surface area, i.e. the surface area on the exterior side of a shell surface of a particle of the invention, the polymer is immobilized (supra). The external surface, area of shell-based particles may be measured by means of techniques known to those skilled in the art such as Atomic Force Microscopy (AFM) and BET isotherm analysis. The external surface area of particles of the invention may range from about 10 to about 500 square meters/gram. The internal surface area, i.e. the surface area facing the interior side of a shell surface of particles of the invention, is in contact with any matter that is included in the void of the particle, such as a pharmaceutically active compound. The internal surface area of shell-based particles with contiguous solid walls cannot be measured directly via techniques such as Atomic Force Microscopy (AFM) and BET isotherm analysis, but can be estimated based on the external particle surface area and particle wall thickness.

The method of covalently coupling a monomer or a polymer to a silica surface may be applied to any silica surface. The silica surface may be the surface of any matter such as a silica coating or a device or structure of silica. In some embodiments the surface is at least essentially smooth. In some embodiments the surface is uneven. In some embodiments the silica surface is the surface of a nanoparticle or a microparticle. Such a nanoparticle or microparticle may include further matter such as a core of a metal or a mixture of metals. In some embodiments the particle is a silica particle. A respective silica particle may in some embodiments have a void and may be a hollow particle (supra).

The monomer or polymer may be attached directly to the silica surface. For example, in case the silica surface carries functional groups (coupling groups) such as —OH, —NH₂ or halogen groups, the direct linkage is possible. As explained above, the monomer or polymer may also optionally be grafted to the silica surface via a bridging group X. Generally, the bridging group may be any molecule that is capable of linking the poly(methacrylic acid) or a copolymer thereof to the silica surface. For example, amino groups or halogen groups may be introduced on the surface of the silica particle, for example, via coupling of an amino-functional or halogen-functional alkylsilane or siloxane, such as for example N-(3-trimethoxysilyl)propyl ethylene diamines (TMSPEA). In one embodiment said bridging group may be according to general Formula (2):

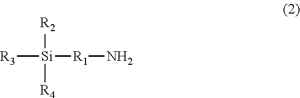

In Formula (2) R₁ is an aliphatic, alicyclic, aromatic or arylaliphatic bridge with a main chain of about 1 to about 10 carbon atoms, such as 1, 2, 3, 4, 5, 6, 7, 8, 9 or 10 carbon atoms. The aliphatic bridge of R₁ may include 0 to about 5 heteroatoms, such as 0 to about 4 heteroatoms, 0 to about 3 or 0 to about 2, e.g. 0, 1, 2, 3, 4 or 5 heteroatoms. These heteroatoms may be selected from the group N, O, S, Se and Si. R₁ may for example be a straight or branched alkyl group. The term “alkyl” refers, unless otherwise stated, to a saturated aliphatic or alicyclic hydrocarbon chain, which may be straight or branched and include heteroatoms. The branches of the hydrocarbon chain may include linear chains as well as non-aromatic saturated cyclic elements. Illustrative examples of non-cyclic alkyl groups are methyl, ethyl, propyl, butyl, pentyl, hexyl, heptyl, octyl, nonyl, decyl, the n isomers of these radicals, isopropyl, isobutyl, isopentyl, neopentyl, sec.-butyl, tert.-butyl, neopentyl and 3,3-dimethylbutyl.

R₂, R₃ and R₄ are, in formula (2) independent from one another, an aliphatic, or alkoxy group with a main chain of about 1 to about 10 carbon atoms such as 1, 2, 3, 4, 5, 6, 7, 8, 9 or 10 carbon atoms. The aliphatic, or alkoxy group may include 0 to about 5 heteroatoms, such as 0 to about 4 heteroatoms, 0 to about 3 or 0 to about 2, e.g. 0, 1, 2, 3, 4 or 5 heteroatoms. These heteroatoms may be selected from the group N, O, S, Se and Si.

In one embodiment of the present invention the bridging group may be, but not limited to, 3-aminoethyl silane, 3-aminopropyl silane (APS), and so on.

The process of providing amino or halogen functional groups on the silica surface may be carried out at ambient temperature or at an elevated temperature, i.e. a temperature above ambient temperature. The temperature may for example be selected in the range from about 30° C. to about 150° C., about 30° C. to about 120° C., about 40° C. to about 100° C. or about 50° C. to about 100° C., e.g. about 50° C., about 60° C., about 70° C., about 80° C., about 90° C. or about 100° C. The process may also be carried out under an inert gas atmosphere, i.e. in the presence of gases that are not reactive, or at least not reactive to a detectable extent, with regard to the reagents and solvents used. Examples of a reactive inert atmosphere are nitrogen or a noble gas such as argon or helium.

In case an amino functional group, has been introduced to the silica surface, it is possible to react such functionalized silica particle with a halogenating agent in order to introduce a halogen group by replacing the amino group. Any common halogenating agent may be used. Examples of halogenating agents may be, but not limited to, N-bromosuccinimide, 2-bromoisobutyryl bromide and so on.

The silica surface that carries amino functional or halogen functional groups is contacted with a carboxy group of the poly(methacrylic acid) or a carboxy group of the methacrylic acid (in case the polymerization is conducted after the linking to the silica surface). The process of coupling the monomer or polymer to the silica surface may in some embodiments be carried out under an inert gas atmosphere (supra). The process may be carried out at room temperature or at an elevated temperature (supra). The temperature may for example be selected in the range from about 30° C. to about 120° C., about 40° C. to about 100° C. or about 50° C. to about 100° C., e.g. about 50° C., about 60° C., about 70° C., about 80° C., about 90° C. or about 100° C. The process may also be carried out under an inert gas atmosphere, i.e. in the presence of gases that are not reactive, or at least not reactive to a detectable extent, with regard to the reagents and solvents used. Examples of a reactive inert atmosphere are nitrogen or a noble gas such as argon or helium. The process may take up to several days, such as about 24 hours, about 36 hours, about 48 hours, about 60 hours or about 72 hours.

The method of the invention is generally carried out in the liquid phase. It may be carried out in any suitable solvent. Any solvent may be used, as long as the (methacrylic acid) or poly(methacrylic acid) or copolymer thereof used dissolves therein sufficiently. Solvents used may be polar or non-polar liquids, including aprotic non-polar liquids. Examples of non-polar liquids include, but are not limited to mineral oil, hexane, heptane, cyclohexane, benzene, toluene, pyridine, dichloromethane, chloroform, carbon tetrachloride, carbon disulfide and a non-polar ionic liquid. Examples of a non-polar ionic liquid include, but are not limited to, 1-ethyl-3-methylimidazolium bis[(trifluoromethyl)-sulfonyl]amide bis(triflyl)amide, 1-ethyl-3-methylimidazolium bis[(trifluoromethyl)sulfonyl]amide trifluoroacetate, 1-butyl-3-methylimidazolium hexafluorophosphate, 1-hexyl-3-methylimidazolium bis(trifluoromethylsulfonyl)imide, 1-butyl-3-methylimidazolium bis(trifluoromethylsulfonyl)imide, trihexyl(tetradecyl)phosphonium bis[oxalato(2-)]borate, 1-hexyl-3-methyl imidazolium tris(pentafluoroethyl)trifluorophosphate, 1-butyl-3-methyl-imidazolium hexafluorophosphate, tris(pentafluoroethyl)trifluorophosphate, trihexyl(tetradecyl)phosphonium; N″-ethyl-N,N,N′,N′-tetramethylguanidinium, 1-butyl-1-methyl pyrroledinium tris(pentafluoroethyl)trifluorophosphate, 1-butyl-1-methyl pyrrolidinium bis(trifluoromethylsulfonyl)imide, 1-butyl-3-methyl imidazolium hexafluorophosphate, 1-ethyl-3-methylimidazolium bis(trifluoromethylsulfonyl)imide and 1-n-butyl-3-methylimidazolium.

In some embodiments the method is carried out in a polar solvent. Examples of a polar solvent include, but are not limited to, dioxane, diethyl ether, diisopropylether, ethylene glycol monobutyl ether, tetrahydrofuran, methyl propyl ketone, methyl isoamyl ketone, methyl isobutyl ketone, cyclohexanone, isobutyl isobutyrate, ethylene glycol diacetate, and a polar ionic liquid. Examples of a polar ionic liquid include, but are not limited to, 1-ethyl-3-methylimidazolium tetrafluoroborate, N-butyl-4-methylpyridinium tetrafluoroborate, 1,3-dialkylimidazolium-tetrafluoroborate, 1,3-dialkylimidazolium-hexafluoroborate, 1-ethyl-3-methylimidazolium bis(pentafluoroethyl)phosphinate, 1-butyl-3-methylimidazolium tetrakis(3,5-bis(trifluoromethylphenyl)borate, tetrabutyl-ammonium bis(trifluoromethyl)imide, ethyl-3-methylimidazolium trifluoromethanesulfonate, 1-butyl-3-methylimidazolium methylsulfate, 1-n-butyl-3-methylimidazolium ([bmim]) octylsulfate, and 1-n-butyl-3-methylimidazolium tetrafluoroborate.

A polar protic solvent can be a solvent that has, for example, a hydrogen atom bound to an oxygen as in a hydroxyl group or a nitrogen as in an amine group. More generally, any molecular solvent which contains dissociable H⁺, such as hydrogen fluoride, is called a protic solvent. The molecules of such solvents can donate an H⁺ (proton). Examples of polar protic solvents include, but are not limited to, water, an alcohol or a carboxylic acid. Examples of an alcohol include, but are not limited to, methanol, ethanol, 1,2-ethanediol (ethylene glycol), 1,3-propanediol, 1,2-propanediol, n-propanol, iso-propanol, n-butanol, iso-butanol, tert-butanol, 2-butanol, 2,3-butanediol (dimethylethylene glycol), 2-methyl-1,3-propanediol, 1-pentanol (amyl alcohol), 2-pentanol, 2-methyl-3-butanol, 3-methyl-1-butanol (iso-pentanol), 3-pentanol (sec-amyl alcohol), 2,4-pentanediol (2,4-amylene glycol), 4-methyl-1,7-heptanediol, 1,9-nonanediol, cyclohexanol, propoxymethanol and 2-ethoxyethanol (ethylene glycol ethyl ether). As four illustrative examples of a carboxylic acid may serve acetic acid, propionic acid, valeric acid and caproic acid.

As mentioned above, the hollow particle of the invention may in some embodiments be water-soluble. Where desired, such a water-soluble microparticle or nanoparticle may be designed for sustained and for controlled delivery. In a sustained system the pharmaceutically active compound is delivered over a prolonged period of time, which overcomes the highly periodic nature of tissue levels associated with conventional (e.g. enteral or parenteral) administration of single doses of free compounds. The term ‘controlled’ indicates that control is exerted over the way in which the pharmaceutically active compound is delivered to the tissues once it has been administrated to the organism to be treated, e.g. the patient.

In the present invention, poly(methacrylic acid)-graft-hollow silica vesicles are obtained. The silica shells provide the stability and poly(methacrylic acid) stealth layers provide pH responsive properties. Thus, the present invention makes use of the fact that poly(methacrylic acid) has different properties depending on the respective pH value of the used system. This means, the structure of the poly(methacrylic acid) shell in aqueous solution depends on the pH value of the medium. At relatively high pH value, the deprotonated poly(methacrylic acid) layers are (fully) soluble in aqueous solution. The pH value of such a solution may be, but not limited to, above 5, such as above 5.5, above 6.0, above 6.5 or above 7.0. In one embodiment the pH value is even above 7.5. Generally, the pH value at which the polymer is soluble may be greater than or equal to 5.0, 5.1, 5.2, 5.3, 5.4, 5.5, 5.6, 5.7, 5.8, 5.9, 6.0, 6.1, 6.2, 6.3, 6.4; 6.5, 6.6, 6.7, 6.8, 6.9, 7.0, 7.1, 7.2, 7.3, 7.4 or 7.5. The solubility of the poly(methacrylic acid) linked to the silica particles become poor at lower pH, for example a pH below 5.0, below 4.5, below 4.0, below 3.5, below 3.0 or below 2.0. Generally, the pH value at which the polymer is less or poor soluble may be less than or equal to 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 or 5.0. Thus, at acidic condition, the shrunk or collapsed PMAA layers seal the pores in the silica shells to prevent the release of the species encapsulated in poly(methacrylic acid)-graft-hollow silica vesicles. “Collapsed” in this respect means that a closed and compact shell is formed when the pH is below about 5.0. Deprotonated PMAA layers become water-soluble so the pores in the silica shells are opened at pH higher than about 6.5 to release the species encapsulated. This mechanism can also be taken form FIG. 6.

As stated above, the hollow silica particles may be used as carrier for pharmaceuticals. Any pharmaceutically active compound may be included into the particle. In some embodiments such a compound is polar and water-soluble. In some embodiments the compound is amphiphilic. In some embodiments the compound is at least essentially non-polar and water-insoluble. The pharmaceutically active compound may be a low molecular weight organic compound. In some embodiments the pharmaceutically active compound is or includes a peptide, a protein, a peptoid, a lipid, a nucleic acid, a saccharide, an oligosaccharide, a polysaccharide or an inorganic molecule. The pharmaceutically active compound may be more or less homogenously distributed, e.g. dispersed, within the hollow microparticle or nanoparticle. In some embodiments the pharmaceutically active compound is located within a certain portion of the water-soluble microparticle or nanoparticle, such as a nanoparticle or the inner wall of a shell of the hollow particle. When provided in a hollow particle, compounds can be protected from the action of components of the ambience such as enzymes, e.g. proteases, in case the pH value is below 5.0. In particular where the particle transiently passes a tissue or organ, e.g. the digestive tract, the particle thereby provides protection from degradation or modification. Nevertheless a compound may be provided in the hollow particle in the form of a prodrug if desired. Further the particle can be used to direct the compound to a desired target or site of action by providing corresponding moieties on the surface of the particle. In such embodiments the application resembles rather a local than a systemic application. Using a particle to encompass a compound also allows the application of a compound that can otherwise hardly be applied via standard application routes, such as an at least essentially non-polar compound. In addition, depending on the selection of the pore size, particle size and other structural features of the particle, the particle provides a diffusion bather as well as a protection from flow and abrupt changes of the ambience. Therefore encompassing matter such as pharmaceutically active compounds in a hollow particle, e.g. a particle with pores, slows the release of matter therefrom. Accordingly, the half-life of compounds in the human or animal body can be controlled by selecting the structural properties of the particle. Typically the half-life of compounds in the human or animal body is longer when applied in a hollow particle.

As used herein, the term “prodrug” means a compound which is converted or released within the human or animal body, e.g. enzymatically, mechanically or electromagnetically, into its active form that has medical effects. A “prodrug” is accordingly a pharmacologically inactive derivative of a parent “drug” molecule. It requires spontaneous or enzymatic biotransformation within the physiological system of the human or animal to which it is administered. “Prodrugs” are commonly used in the art to overcome problems associated with stability, toxicity, lack of specificity, or limited bioavailability. They often offer advantages of solubility, tissue compatibility, or delayed release in the mammalian organism. As an illustrative example, a “prodrug” may be a low molecular weight compound with a protective group shielding a moiety or functional group thereof and thereby reversibly suppressing the activity of the functional group. A respective “prodrug” may become pharmaceutically active in vivo or in vitro when the protective group undergoes solvolysis or enzymatic removal. As a further illustrative example, a functional group may only be introduced into a compound of general formula (1) upon biochemical transformation such as oxidation, phosphorylation, or glycosylation. Thus a respective “prodrug” may only be converted into a compound of general formula (1) by an enzyme, gastric acid, etc. in the human or animal body. The “prodrug” of a compound of general formula (1) may be a hydrate or a non-hydrate. Common “prodrugs” include acid derivatives such as esters prepared by reaction of parent acids with a suitable alcohol (e.g., a lower alkanol), amides prepared by reaction of the parent acid compound with an amine (e.g., as described above), or basic groups reacted to form an acylated base derivative (e.g., a lower alkylamide).

In this regard the present invention also provides: a pharmaceutical composition. The pharmaceutical composition includes one or more hollow particles that have a pharmaceutically active compound in the void of the hollow particle. As detailed above, the void may be encompassed by a shell that surrounds the void. The particles that are included in the pharmaceutical composition are typically water-soluble. Generally a particle is rendered water-soluble by selecting an appropriate polar or amphiphilic polymer for immobilization thereon.

In some embodiments a respective particle, e.g. a water-soluble particle, of the invention is coupled to a molecule with binding affinity for a selected target tissue or for a selected target molecule, such as a microorganism, a virus particle, a peptide, a peptoid, a protein, a nucleic acid, a peptide, an oligosaccharide, a polysaccharide, an inorganic molecule, a synthetic polymer, a small organic molecule or a drug.

Illustrative examples of a molecule with binding affinity for a certain target are an antibody, a fragment thereof and a proteinaceous binding molecule with antibody-like functions. Examples of (recombinant) antibody fragments are Fab fragments, Fv fragments, single-chain Fv fragments (scFv), diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). An example of a proteinaceous binding molecule with antibody-like functions is a mutein based on a polypeptide of the lipocalin family (WO 03/029462, Beste et al., Proc. Natl. Acad. Sci. U.S.A. (1999) 96, 1898-1903). Lipocalins, such as the bilin binding protein, the human neutrophil gelatinase-associated lipocalin, human Apolipoprotein D or glycodelin, posses natural ligand-binding sites that can be modified so that they bind to selected small protein regions known as haptens. Examples of other proteinaceous binding molecules are the so-called glubodies (see e.g. internation patent application WO 96/23879), proteins based on the ankyrin scaffold (Mosavi, L. K., et al., Protein Science (2004) 13, 6, 1435-1448) or crystalline scaffold (e.g. internation patent application WO 01/04144) the proteins described in Skerra, J. Mol. Recognit. (2000) 13, 167-187, AdNectins, tetranectins and avimers. Avimers contain so called A-domains that occur as strings of multiple domains in several cell surface receptors (Silverman, J., et al., Nature Biotechnology (2005) 23, 1556-1561). Adnectins, derived from a domain of human fibronectin, contain three loops that can be engineered for immunoglobulin-like binding to targets (Gill, D. S. & Damle, N. K., Current Opinion in Biotechnology (2006) 17, 653-658). Tetranectins, derived from the respective human homotrimeric protein, likewise contain loop regions in a C-type lectin domain that can be engineered for desired binding (ibid.). Peptoids, which can act as protein ligands, are oligo(N-alkyl) glycines that differ from peptides in that the side chain is connected to the amide nitrogen rather than the □ carbon atom. Peptoids are typically resistant to proteases and other modifying enzymes and can have a much higher cell permeability than peptides (see e.g. Kwon, Y.-U., and Kodadek, T., J. Am. Chem. Soc. (2007) 129, 1508-1509).

As a further illustrative example, a linking moiety such as an affinity tag may be used to immobilise a molecule with binding affinity for a selected target tissue or for a selected target molecule. Such a linking moiety may be a molecule, e.g. a hydrocarbon-based (including polymeric) molecule that includes nitrogen-, phosphorus-, sulphur-, carben-, halogen- or pseudohalogen groups, or a portion thereof. As an illustrative example, the silica surface may include functional groups. Such functional groups may be residual groups, e.g. amino groups, used and/or provided for the covalent attachment of the polymer, and which did not undergo a coupling reaction therewith. These groups may allow for the covalent attachment of a biomolecule, for example a molecule such as a protein, a nucleic acid molecule, a polysaccharide or any combination thereof.

Examples of an affinity tag include, but are not limited to biotin, dinitrophenol or digoxigenin, oligohistidine, polyhistidine, an immunoglobulin domain, maltose-binding protein, glutathione-S-transferase (GST), calmodulin binding peptide (CBP), FLAG′-peptide, the T7 epitope (Ala-Ser-Met-Thr-Gly-Gly-Gln-Gln-Met-Gly), maltose binding protein (MBP), the HSV epitope of the sequence Gln-Pro-Glu-Leu-Ala-Pro-Glu-Asp-Pro-Glu-Asp of herpes simplex virus glycoprotein D, the hemagglutinin (HA) epitope of the sequence Tyr-Pro-Tyr-Asp-Val-Pro-Asp-Tyr-Ala, the “myc” epitope of the transcription factor c-myc of the sequence Glu-Gln-Lys-Leu-Ile-Ser-Glu-Glu-Asp-Leu, or an oligonucleotide tag. Such an oligonucleotide tag may for instance be used to hybridise to an immobilised oligonucleotide with a complementary sequence. A further example of a linking moiety is an antibody, a fragment thereof or a proteinaceous binding molecule with antibody-like functions (see also above).

A further example of a linking moiety is a cucurbituril or a moiety capable of forming a complex with a cucurbituril. A cucurbituril is a macrocyclic compound that includes glycoluril units, typically self-assembled from an acid catalyzed condensation reaction of glycoluril and formaldehyde. A cucurbit[n]uril, (CB[n]), that includes n glycoluril units, typically has two portals, with polar ureido carbonyl groups. Via these ureido carbonyl groups cucurbiturils can bind ions and molecules of interest. As an illustrative example cucurbit[7]uril (CB[7]) can form a strong complex with ferrocenemethylammonium or adamantylammonium ions. Either the cucurbit[7]uril or e.g. ferrocenemethylammonium may be attached to a biomolecule, while the remaining binding partner (e.g. ferrocenemethylammonium or cucurbit[7]uril respectively) can be bound to a selected surface. Contacting the biomolecule with the surface will then lead to an immobilisation of the biomolecule. Functionalised CB[7] units bound to a gold surface via alkanethiolates have for instance been shown to cause an immobilisation of a protein carrying a ferrocenemethylammonium unit (Hwang, I., et al., J Am. Chem. Soc. (2007) 129, 4170-4171).

Further examples of a linking moiety include, but are not limited to an oligosaccharide, an oligopeptide, biotin, dinitrophenol, digoxigenin and a metal chelator (cf. also below). As an illustrative example, a respective metal chelator, such as ethylenediamine, ethylenediaminetetraacetic acid (EDTA), ethylene glycol tetraacetic acid (EGTA), diethylenetriaminepentaacetic acid (DTPA), N,N-bis(carboxymethyl)glycine (also called nitrilotriacetic acid, NTA), 1,2-bis(o-aminophenoxy)ethane-N,N,N′,N′-tetraacetic acid (BAPTA), 2,3-dimercapto-1-propanol (dimercaprol), porphine or heme may be used in cases where the target molecule is a metal ion. As an example, EDTA forms a complex with most monovalent, divalent, trivalent and tetravalent metal ions, such as e.g. silver (Ag+), calcium (Ca2+), manganese (Mn2+), copper (Cu2+), iron (Fe2+), cobalt (Co3+) and zirconium (Zr4+), while BAPTA is specific for Ca2+. In some embodiments a respective metal chelator in a complex with a respective metal ion or metal ions defines the linking moiety. Such a complex is for example a receptor molecule for a peptide of a defined sequence, which may also be included in a protein. As an illustrative example, a standard method used in the art is the formation of a complex between an oligohistidine tag and copper (Cu2+), nickel (Ni2+), cobalt (Co2+), or zink (Zn2+) ions, which are presented by means of the chelator nitrilotriacetic acid (NTA). Avidin or streptavidin may for instance be employed to immobilise a biotinylated nucleic acid, or a biotin containing monolayer of gold may be employed (Shumaker-Parry, J. S., et al., Anal. Chem. (2004) 76, 918).

A molecule or moiety immobilized on the porous particulate material may also serve in cross-linking individual particles. An organic bridge may for instance be formed between two particles by a phenylene-bridge or an ethylene-bridge. The bridge may also include a chiral moiety such as bulk vanadyl Schiff base complexes, a binaphthyl group, a 1,2-diaminocyclohexane group, a trans-(1R,2R)-bis-(ureido)-cyclohexyl-bridge (Zhu, G., et al., Microporous and Mesoporous Materials (2008) 116, 36-43) or a chiral borated ethylene-bridge. The resulting particulate matter is a chiral porous material with chiral induction ability in e.g. asymmetric catalysis. A further example of a chiral moiety that may be immobilized on the porous particulate material is a carbohydrate, including a cellulose derivative such as cellulose tris(3,5-dimethylphenyl carbamate).

A particulate porous metal oxide or metalloid oxide obtainable according to the present invention may be used as a catalyst or as a support for a catalyst. In some embodiments it may for instance be used in catalytic combustion, such as the oxidation of volatile organic compounds, e.g. propen “(Orlov & Klinowski, 2009, supra). In embodiments where the particulate porous material according to the invention includes more than one metal oxide or metalloid oxide one or more, including all, of the respective oxides may show a corresponding catalytic activity. In CuO-loaded mesoporous SBA-15 the copper oxide may for instance serve as the catalyst in the hydroxylation of benzene to phenol Kong et al., 2009, supra). A particulate porous metal oxide or metalloid oxide obtainable according to the invention may also be functionalized with chelating ligands, to which catalytically active organometallic complexes may be complexed. As an illustrative example, diphenylphosphino ligands may be covalently bound to the porous metal or metalloid oxide, and Pd— or Ru-containing organometallic silanes chelated thereto as described by Zhang et al. (Advanced Functional Materials (2008) 18, 3590-3597). Multiple active sites may be introduced, thereby forming a multifunctional catalyst (ibid.).

The present invention also relates to the use of hollow silica particles in e.g. the separation of a mixture of molecules in a fluid, in catalysis, in nonlinear optics, as an ion-exchange coating, in the formation of a solid-state electrochemical device and in the formation of a drug delivery vehicle. Since silica has high biocompatibility and at the same time mechanical strength, thermal and pH stability, a large variety of applications are possible.

In some embodiments micro- or nanoparticles of the invention can be used in the separation of a mixture of molecules in a fluid such as chromatography, e.g. as gas chromatography, capillary electrochromatography, HPLC (high performance liquid chromatography) or UPLC (ultrahigh pressure liquid chromatography). In such separation applications a plurality of the hollow micro- or nanoparticles is typically used as a chromatography stationary phase.

As indicated above, particles according to the invention can be used as a carrier for a drug, a marker or other matter to be administered to a human or animal body. The micro- or nanoparticles described herein, as well as matter such as compounds included therein, can be administered to a cell, an animal or a human patient per se, or in a pharmaceutical composition. In a pharmaceutical composition the particles may be mixed with other active ingredients, as in combination therapy, or with suitable carriers or excipient(s). Techniques for formulation and administration of respective particles resemble or are identical to those of low molecular weight compounds well established in the art. Exemplary routes include, but are not limited to, oral, transdermal, and parenteral delivery. A plurality of the particles may be used to fill a capsule or tube, or may be compressed as a pellet. The micro- or nanoparticles may also be used in injectable or sprayable form, for instance as a suspension or in a gel formulation.

The pharmaceutical composition of the present invention including the hollow silica particles offers great advantages for the administration of pharmaceutical compounds. Due to the pH sensitivity, the encapsulated, drug will not be released, for example, by the gastric acid (pH below about 5.0) in the stomach, i.e. no significant drug leakage in the stomach can be observed as the poly(methacrylic acid) is “collapsed” and the pores of the silica core are closed. The drug is protected so that no degradation or modification of the active compound will take place. Once the drug passes to the intestine, the pH value of the environment raises to above about 5.0 or more. As explained above, the poly(methacrylic acid) chains will be soluble then and the pores of the shell will open. Thus, the drug can be released.

In one embodiment the silica particles of the invention may be used to formulate various drug systems, especially for oral delivery of drugs. The pH responsive properties of PMAA-graft-hollow silica vesicles may, for example, be very suitable for delivery of sensitive drugs, for example, proteinaceous drugs such as insulin or acid labile drugs. In the acidic stomach, the shrunk PMAA layers will protect such drugs, for example, insulin from degradation by enzyme, and in intestine, the fully soluble deprotonated PMAA layers will make it easy to release insulin. PMAA-graft-hollow silica vesicles may, for example, also be applied to formulate immunosupressants such as cyclosporine A to thereby provide more predictable bioavailability of cyclosporine A.

Suitable routes of administration may, for example, include depot, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular, injections. It is noted in this regard that for administering micro- or nanoparticles a surgical procedure is not required. The route of administration depends on the one hand on the pharmaceutical used and on the other hand on the conditions present at the administration site. This means, the conditions at the administration site or at the site the pharmaceutical is delivered have to be in such a way that the poly(methacrylic acid) chains are soluble and the pores of the silica vesicle are open. Generally, the poly(methacrylic acid) may be further adapted by substituting the side chains in order to achieve the respective properties. In one embodiment the administration route oral.

Alternately, one may administer the particles in a local rather than systemic mariner, for example, via injection of the compound directly into a solid tumour, often in a depot or sustained release formulation.

Pharmaceutical compositions that include the particles of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers including excipients and auxiliaries that facilitate processing of the particles into preparations that can be used pharmaceutically. Proper formulation is dependent upon the route of administration chosen.

For injection, the particles of the invention may be formulated in aqueous solutions, for instance in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the micro- or nanoparticles can be formulated readily by combining them with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatine, as well as soft, sealed capsules made of gelatine and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the particles may be suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

The micro- or nanoparticles may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical compositions suitable for use in the present invention include compositions where the active ingredients included in the micro- or nanoparticles are contained in an amount effective to achieve its intended purpose. Determination of a therapeutically effective amount is well within the capability of those skilled in the art, especially in light of the detailed disclosure provided herein.

For any compound used in the micro- or nanoparticles of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the kinase activity). Such information can be used to more accurately determine useful doses in humans.

The micro- or nanoparticles may, if desired, be presented in a pack or dispenser device which may contain one or more unit dosage forms containing the particles with the active ingredient. The pack may for instance include metal or plastic foil, such as a blister pack. The pack or dispenser device may be accompanied by instructions for administration. The pack or dispenser may also be accompanied with a notice associated with the container in a form prescribed by a governmental agency regulating, the manufacture, use, or sale of pharmaceuticals, which notice is reflective of approval by the agency of the form of the compound for human or veterinary administration. Such notice, for example, may be the labeling approved by the U.S. Food and Drug Administration or other government agency for prescription drugs, or the approved product insert.

In order that the invention may be readily understood and put into practical effect, particular embodiments will now be described by way of the following non-limiting examples. It is understood that modification of detail may be made without departing from the scope of the invention.

### EXEMPLARY EMBODIMENTS OF THE INVENTION

Exemplary embodiments of methods according to the invention as well as reactants and further processes that may be used are shown in the appending figures.

### EXAMPLE 1

### Preparation of Hollow Silica Spheres

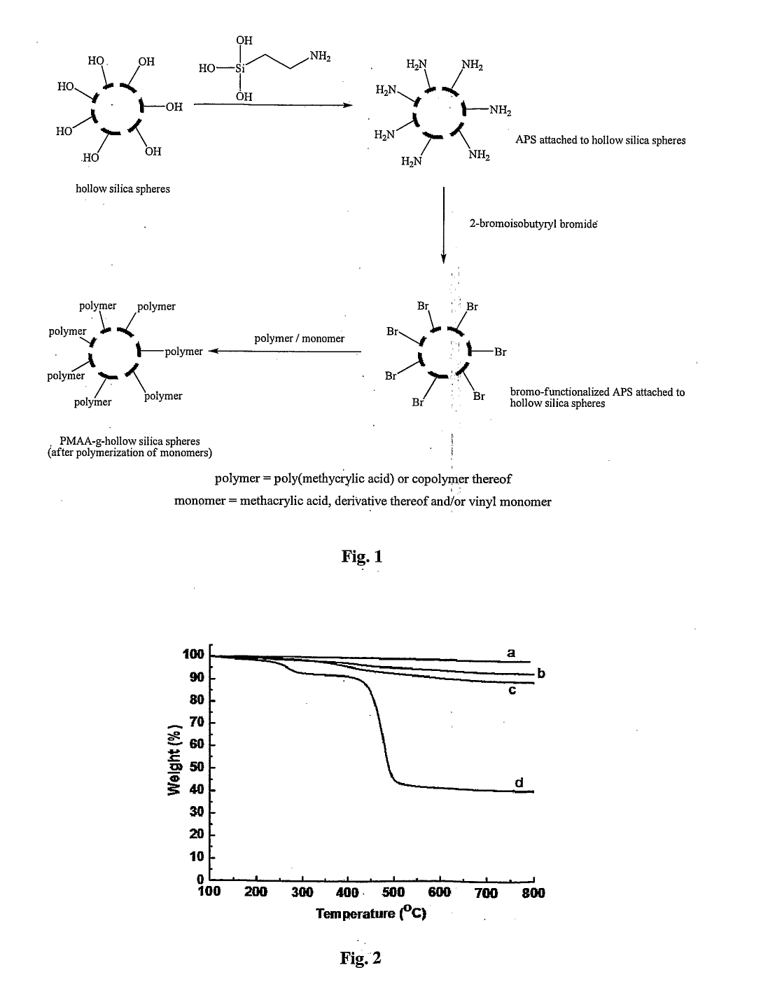

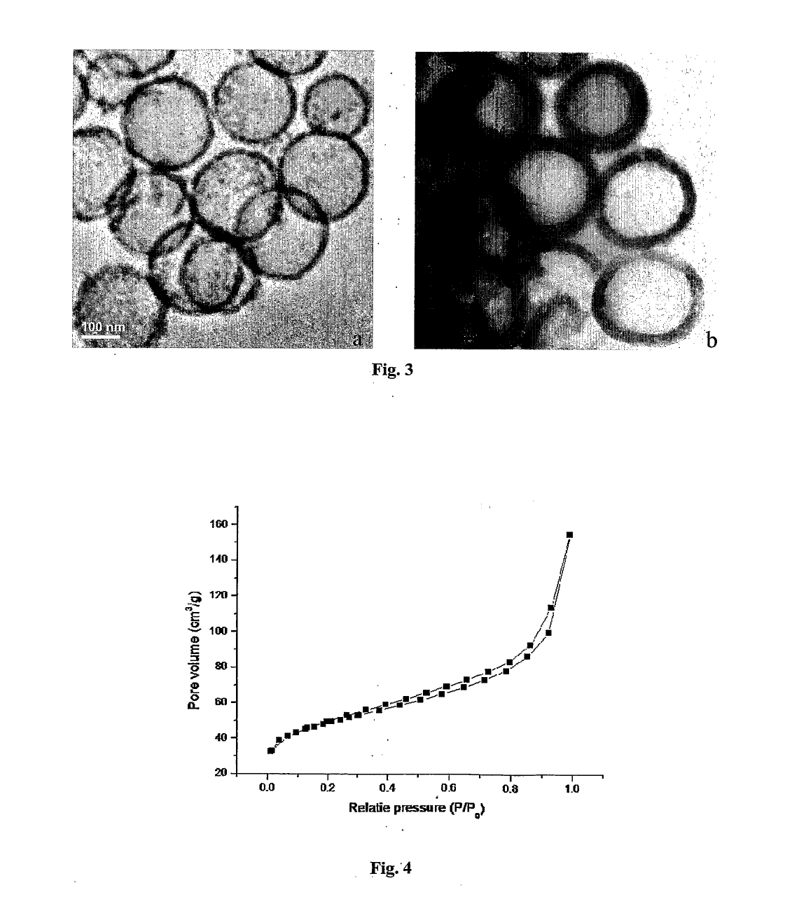

Hollow silica can be prepared using polystyrene colloids as templates of sol-gel reaction of tetraethoxysilane (TEOS) followed by calcination. In a typical process, 3.0 g of poly(vinyl-pyrrolidone) (PVP) (Mw: 40 K) was dissolved in 100 mL of HPLC grade water under stirring for 24 hr at room temperature. Then 11.0 mL of styrene and 0.26 g of α,α′-azodiisobutyramidine dihydrochloride (AIBA) were added to the solution under stirring at 100 rpm and 70° C. under Argon. After 24 h, polystyrene colloid solution was obtained. 18 mL of polystyrene colloid solution was mixed with 240 mL of ethanol and 12 mL ammonia solution (NH₄OH) (25 wt %). Then 3.18 mL of TEOS in 5 mL of ethanol was added dropwise, and the mixture was stirred at 50° C. for 24 h. The solid was collected by centrifugation and calcinated at 550° C. to get hollow silica particles. FIG. 1a is TGA curve of the obtained hollow silica, and FIG. 2a is TEM images of hollow silica. FIG. 3 is the nitrogen adsorption-desorption isotherms of the hollow silica obtained. The average diameter of the pores in the silica shells is 1.49 nm. The surface area and the total pore volume of hollow silica are 193 m²/g and 0.21 cm³/g obtained using the BET and BJH methods, respectively.

### EXAMPLE 2

### Preparation of Hollow Silica Particles Functionalized with Amino Groups (HSilica-NH₂)

Typically 2 g of hollow silica was dispersed in 90 mL of p-xylene. After 3 mL of 3-aminopropyl silane (APS) was added, the solution was stirred at 90° C. under Argon for 24 h. The solution was precipitated in ether. The solid was collected by filtration and was purified by washing with ether. FIG. 1b is the TGA curve of HSilica-NH₂.

### EXAMPLE 3

### Preparation of Hollow Silica Attached with Initiator (HSilica-Br)

1.9 g of HSilica-NH₂ was dispersed in 60 mL of dry chloroform in a 100 mL flask. After 1.7 mL of triethylamine was added into the solution, the flask was immersed in an ice-water bath, and 0.6 mL of 2-bromoisobutyryl bromide in 5 mL of chloroform was added dropwise. 1.5 h later, the flask was taken out and was stirred at ambient temperature for 4 h. Then the solution was filtrated through PTFE membranes with pores of diameter of 0.2 μm. The solid collected was washed with fresh chloroform for 5 times followed by drying under vacuum at 50° C. FIG. 1c is the TGA curve of HSilica-Br.

### EXAMPLE 4

### Preparation of Poly(Methacrylic Acid)-Grafted-Hollow Silica (PMAA-g-Hollow Silica) Vesicles

0.5 g of HSilica-Br was dispersed in 18 mL of HPLC water. Then 7 g of sodium methacrylic acid. 0.043 g of CuBr₂, 0.138 g of Curb, and 0.347 g of 2,2′-bipyridine were added into the solution. After three cycles of freeze-vacuum-thaw were performed under Argon, the mixture was stirred under ambient temperature for 3 days. The mixture was dispersed in 200 mL of DI water, and pH of the solution was adjusted to pH 2. The solution was centrifuged. The solid collected was washed with methanol for 6 times followed by drying under vacuum at 50° C.

### EXAMPLE 5

### Characterization of PMAA-g-Hollow Silica

The amounts of PMAA in PMAA-g-hollow silica were measured using TGA. FIG. 1d shows the TGA curves of PMAA-g-hollow silica. From FIG. 1, the content of PMAA in PMAA-g-hollow silica is determined to be 48% (w/w). FIG. 2b are TEM images PMAA-g-hollow silica. In comparison with TEM images of pristine hollow silica as shown in FIG. 2a, PMAA stealth layers of PMAA-g-hollow silica vesicles can be observed obviously.

### EXAMPLE 6

### pH Responsive Shrinking of PMAA Brushes in PMAA-g-Hollow Silica

¹H NMR spectra of PMAA-g-hollow silica at pH 3.4 and pH 7.4 respectively are shown in FIG. 4. PEG was used as an external reference. The peaks attributed to the protons of PMAA brushes are broader at pH 3.4 than at pH 7.4. When pH was adjusted from 3.4 to 7.4, the peaks shift upfield, and the ratio of the integrity intensity of proton peaks of PMAA brushes to those of external reference PEG increased from 1.1 to 2.0. This indicates that PMAA brushes have a poor solubility in aqueous solution at pH 3.4 than at pH 7.4. The increased solubility of PMAA brushes at pH 7.4 is due to deprotonation of PMAA. So the PMAA brush segments become shrunk at pH 3.4 but soluble at pH 7.4, and this property make PMAA suitable as pH responsive gates of the nanosized pores in the silica shells for controlled release.

### EXAMPLE 7

### pH Responsive Release of Calcein Blue

To load Calcein blue into PMAA-g-hollow silica, 25 mg of PMAA-g-hollow silica was dispersed in 2.5 mL of DI H₂O with pH adjusted to pH 7.5, then 1.0 mg of Calcein blue was dissolved in the solution. After the solution was shaken for 24 h, pH of the solution was adjusted to pH 2. Calcein blue loaded PMAA-g-hollow silica was collected by centrifugation and washed with fresh pH 2 DI water for three times to remove unloaded Calcein blue.

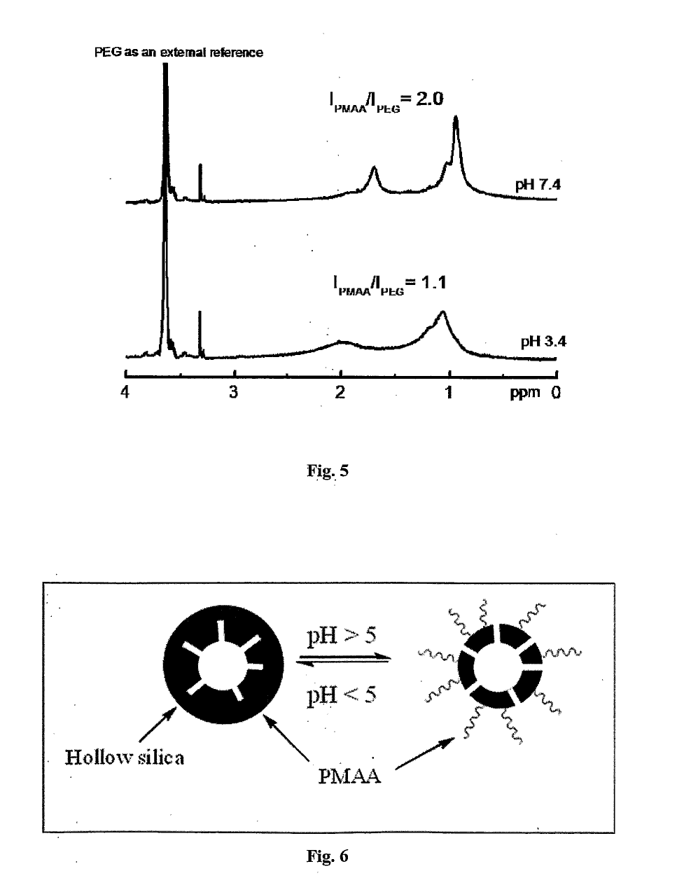

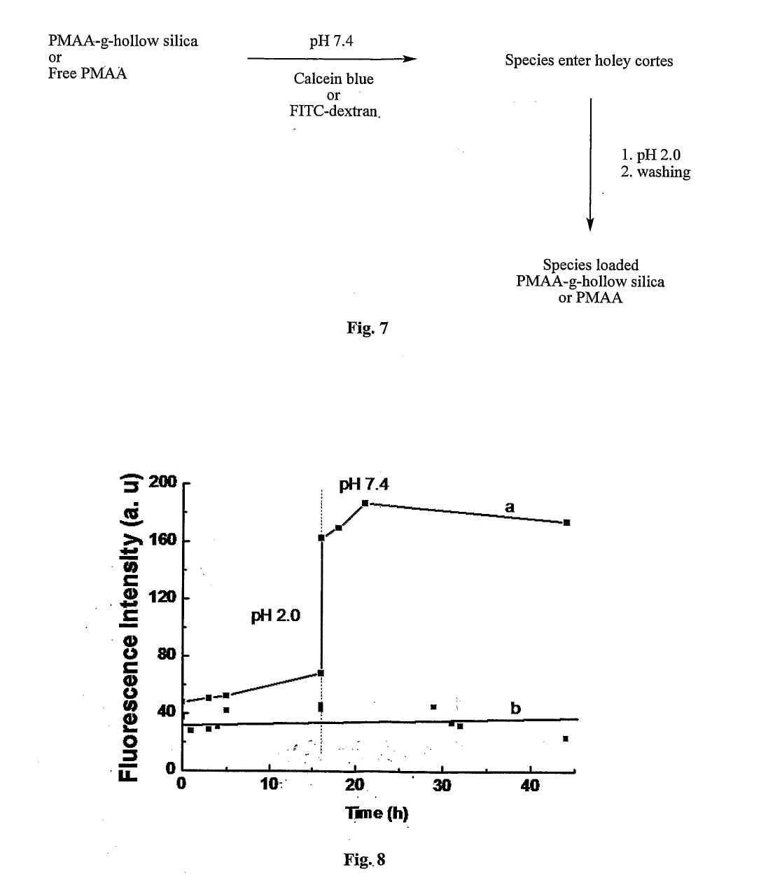

To measure the release profile of Calcein blue, Calcein blue loaded PMAA was dispersed in 50 mL of pH 2.0 DI water. The release profile at pH 2.0 was monitored first. At designed time intervals, 1.5 mL of the solution was taken out and diluted with 3.5 mL of pH 2.0 DI water. The mixture was filtered through membranes with pores of diameter of 0.22 μm to separate PMAA-g-hollow silica. Then the fluorescence intensity of the filtrate was measured at 437 nm with an irradiation at 322 nm. After the release profile at acid condition was measured, the pH of the solution was adjusted to 7.5, and the release profile was measures similarly. The results are presented in FIG. 5.

In order to evaluate the interaction of free PMAA with Calcein blue, a controlled experiment was performed. 25 mg of free PMAA (Mw: 100 k) was dispersed in 2.5 mL of DI H₂O with pH adjusted to pH 7.5, then 1.0 mg of Calcein blue was mixed with the solution. After the solution was shaken for 24 h, pH of the solution was adjusted to pH 2. The solid was collected by centrifugation filtration through membranes with molecular weight cutting of 30 K to remove free Calcein blue followed by washing with fresh pH 2 DI water for three times. To measure the release profile of Calcein blue, the solid obtained was dispersed in 50 mL of pH 2.0 DI water. The release profile at pH 2.0 was monitored first. At designed time intervals, 1.5 mL of the solution was taken out and diluted with 3.5 mL of pH 2.0 DI water. The mixture was filtrated by centrifugation through membranes with molecular weight cutting of 30 K to separate PMAA. Then the fluorescence intensity of the filtrate was measured at 437 nm with an irradiation at 322 nm. After the release profile at acid condition was measured, pH of the solution was adjusted to 7.5, and the release profile was measured similarly. The results are presented in FIG. 8 also.

FIG. 8 shows that the fluorescence intensity of Calcein blue released from free PMAA change insignificantly with time and pH. In contrast, the fluorescence intensity of Calcein blue released from PMAA-g-hollow silica vesicles increases very slowly at pH 2.0 but an abrupt increase was observed when pH was adjusted to pH 7.5. These results reflect that Calcein blue was released slowly at pH 2.0 but much fast at pH 7.5 from PMAA-g-hollow silica vesicles. Calcein blue was loaded into PMAA-g-hollow silica vesicles by mixing with aqueous solution of PMAA-g-hollow silica at pH 7.5. Under this condition, deprotonated PMAA brushes are fully soluble in aqueous solution so the pores in the silica shells are accessible to Calcein blue molecules. When pH of the solution was adjusted to pH 2, PMAA brushes became shrunk and the pores in the silica shells were sealed so Calcein blue was encapsulated in the holey cores. In the release process, shrunk PMAA brushes cover the pores in the silica shells at pH 2.0 so Calcein blue is released from PMAA-g-hollow silica vesicles slowly. However, at pH 7.5, deprotonated PMAA brushes become fully soluble in aqueous solution and the pores in the silica shells are opened, so Calcein blue is released fast. However, just free PMAA cannot provide this pH responsive property without holey cores presented in PMAA-g-hollow silica.

### EXAMPLE 8

### pH Responsive Release of Fluoresceinylisothiocyanato-Dextran (FITC-Dextrane) (Mw: 10 K)

To load FITC-dextrane (Mw: 10 K) into PMAA-g-hollow silica, 25 mg of PMAA-g-hollow silica was disperse in 2.5 mL of DI H₂O with pH adjusted to pH 7.5, then 1.0 mg of FITC-dextrane (Mw: 10 K) was dissolved in the solution. After the solution was shaken for 24 h, pH of the solution was adjusted to pH 2. FITC-dextrane (Mw: 10 K) loaded PMAA-g-hollow silica was collected by centrifugation and washed with fresh pH 2 DI water for 3 times to remove unloaded FITC-dextrane (Mw: 10 K).

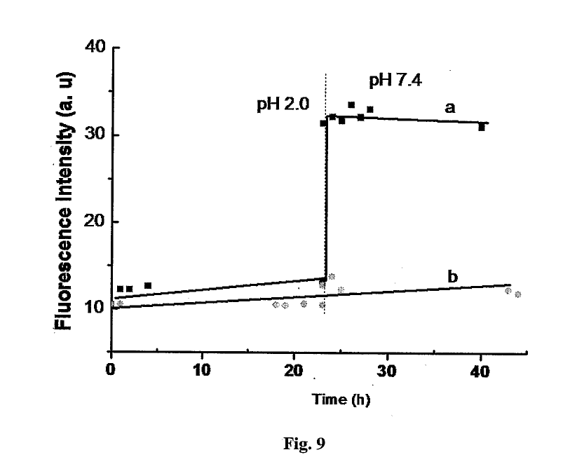

To measure the release profile of FITC-dextran (Mw: 10 K), FITC-dextrane (Mw: 10 K) loaded PMAA was dispersed in 50 mL of pH 2.0 DI water. The release profile at pH 2.0 was monitored first. At designed time intervals, 1.5 mL of the solution was taken out and diluted with 3.5 mL of pH 2.0 DI water. The mixture was filtered through membranes with pores of diameter of 0.22 μm to separate PMAA-g-hollow silica. Then the fluorescence intensity of the filtrate was measured at 510 nm with an irradiation at 440 nm. After the release profile at acid condition was measured, pH of the solution was adjusted to 7.5, and the release profile was measured similarly. The results are presented in FIG. 9.

In order to evaluate the interaction of free PMAA with FITC-dextrane (Mw: 10 K), a controlled experiment was performed. 25 mg of free PMAA (Mw 100 k) was dispersed in 2.5 mL of DI H₂O with pH adjusted to pH 7.5, then 1.0 mg of FTIC-dextrane (Mw: 10 K) was mixed with the solution. After the solution was shaken for 24 h, pH of the solution was adjusted to 2. The solid was collected by centrifugation filtration through membranes with a molecular weight cutting of 30 K to remove free FITC-dextrane (Mw: 10 K). The solid was washed with fresh pH 2 DI water for 3 times. To measure the release profile of FITC-dextrane (Mw: 10 K), the solid obtained was dispersed in 50 mL of pH 2.0 DI water. The release profile at pH 2.0 was monitored first. At designed time intervals, 1.5 mL of the solution was taken out and diluted with 3.5 mL of pH 2.0 DI water. The mixture was filtrated by centrifugation through membranes with a molecular weight cutting of 30 K to separate PMAA. Then the fluorescence intensity of the filtrate was measured at 510 nm with an irradiation at 440 nm. After the release profile at acid condition was measured, pH of the solution was adjusted to 7.5, and the release profile was measured similarly. The results are presented in FIG. 9 also.

FIG. 9 shows that the fluorescence intensity of FITC-dextran (Mw: 10 K) released from free PMAA change insignificantly with time and pH. In contrast, the fluorescence intensity of FITC-dextran (Mw: 10 K) released from PMAA-g-hollow silica vesicles increases very slowly at pH 2.0 but an abrupt increase was observed when pH was adjusted to pH 7.5. These results reflect that FITC-dextrane (Mw: 10 K) was released slowly at pH 2.0 but much fast at pH 7.5 from PMAA-g-hollow silica vesicles. Here FITC-dextrane (Mw: 10 K) was loaded into PMAA-g-hollow silica vesicles by mixing with aqueous solution of PMAA-g-hollow silica at pH 7.5. Under this condition, deprotonated PMAA brushes are fully soluble in aqueous solution so the pores in the silica shells are accessible to FITC-dextran (Mw: 10 K) molecules. When pH of the solution was adjusted to pH 2, PMAA brushes became shrunk and the pores in the silica shells were sealed so FITC-dextran (Mw: 10 K) was encapsulated in the holey cores. In the release process, shrunk PMAA brushes cover the pores in the silica shells at pH 2.0 so FITC-dextrane (Mw: 10 K) was released from PMAA-g-hollow silica vesicles slowly. However, at pH 7.5, deprotonated PMAA brushes become fully soluble in aqueous solution and the pores in the silica shells are opened, so FITC-dextrane (Mw: 10 K) is released fast. However, just free PMAA cannot provide this pH responsive property without holey cores presented in PMAA-g-hollow silica.

In comparison with Calvein blue, FITC-dextrane (Mw: 10 K) with a higher molecular weight still can be loaded into PMAA-g-hollow silica vesicles, and pH responsive release of FITC-dextrane (10 K) can be obtained.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognised that various modifications are possible within the scope of the invention claimed. Additional objects, advantages, and features of this invention will become apparent to those skilled in the art upon examination of the foregoing examples and the appended claims. Thus, it should be understood that although the present invention is specifically disclosed by exemplary embodiments and optional features, modification arid variation of the inventions embodied therein herein disclosed may be resorted to by those skilled in the art, and that such modifications and variations are considered to be within the scope of this invention. In addition, where features or aspects of the invention are described in terms of Markush groups, those skilled in the art will recognise that the invention is also thereby described in terms of any individual member or subgroup of members of the Markush group.

1-20. (canceled)

21. A porous hollow silica particle with a polymer grafted thereon, wherein the polymer is selected from poly(methacrylic acid) and copolymers thereof, wherein the polymer is grafted to the silica particle via a bridging group selected from an aminoalkyl silane, halogenalkyl silane and an aminoalkoxysilane.

22. The silica particle of claim 21, wherein the poly(methacrylic acid) is of the general formula (1) 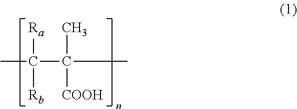
wherein Rₐ and R_{b} are independently an aliphatic, an alicyclic, an aromatic or an arylaliphatic group with a main chain of about 1 to about 30 carbon atoms and 0 to about 10 heteroatoms selected from the group consisting of N, O, S, Se and Si; and n is an integer from 2 to 10000; wherein all groups may be optionally substituted.

23. The silica particle of claim 21, wherein the copolymer is selected from compounds of formula (1) and vinyl monomers of the general formula (3) CH₂═CRₓR_{y}, wherein in formula (3) Rₓ and R_{y} are each independently selected from the group consisting of H, optionally substituted aliphatic, an alicyclic, an aromatic and an arylaliphatic group with a main chain of about 1 to about 30 carbon atoms and 0 to about 10 heteroatoms selected from the group consisting of N, O, S, Se and Si.

24. The silica particle of claim 21, wherein the surface of the silica particle has a polymer content in the range from about 10% to about 90% (w/w).

25. The silica particle of claim 24, wherein the surface of the silica particle has a polymer content in the range of about 30% to about 80% (w/w).

26. The silica particle of claim 21, wherein the silica particle has a maximal width of about 1 nm to about 100 μm.

27. The silica particle of claim 26, wherein the silica particle has a maximal width of about 10 nm to about 10 μm.

28. The silica particle of claim 21, wherein the particle is microporous or mesoporous.

29. The silica particle of claim 21, wherein the average diameter of the pores of the particles is between about 1 nm to about 50 nm.

30. The silica particle of claim 21, wherein the hollow particle has an inner void that comprises a pharmaceutically active compound.

31. The silica particle of claim 21, wherein the pores of the particle are sealed at a pH <about 5.

32. The silica particle of claim 21, wherein the pores of the particle are open at a pH >about 5.

33. A pharmaceutical composition comprising a plurality of porous hollow silica particles according to claim 21.

34. A method of preparing a porous hollow silica particle with a polymer grafted thereon, the method comprising:
providing a porous hollow silica particle having a silica surface;
providing an aminosilane compound according to general formula (2), 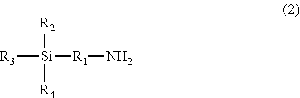
wherein R₁ is one of an aliphatic, an alicyclic, an aromatic and an arylaliphatic bridge with a main chain of about 1 to about 10 carbon atoms and 0 to about 5 heteroatoms selected from the group consisting of N, O, S, Se and Si, and R₂, R₃ and R₄ are independently selected from an aliphatic group and an alkoxy group with a main chain of about 1 to about 10 carbon atoms and 0 to about 3 heteroatoms selected from the group N, O, S, Se and Si;
optionally reacting the amino silane with a halogenating agent;
contacting the compound of the general Formula (2) and the silica surface;
allowing at least one of the groups R₂, R₃ and R₄ of the compound of the general Formula (2) to undergo a coupling reaction with the silica surface, thereby forming a covalent bond with the same;
providing a monomer selected from methacrylic acid and derivatives thereof or a polymer selected from poly(methacrylic acid) and derivatives thereof;
contacting the monomer or the polymer and the silica surface;
allowing the carboxy group of the monomer or the polymer and the amino functional group or the halogen functional group on the silica surface to undergo a coupling reaction, thereby covalently coupling the monomer or the polymer to the silica surface; and
optionally polymerizing the monomer with further monomers.

35. The method of claim 34, wherein the silica surface is the surface of a micro- or nanoparticle.

36. The method of claim 34, wherein the halogenating agent is 2-bromoisobutyryl bromide.
